(19) United States
(12) Patent Application Publication
(10) Pub. No.: US 2024/0252464 A1
(43) Pub. Date: Aug. 1, 2024

(54) A SELF-EMULSIFYING DRUG DELIVERY FORMULATION WITH IMPROVED ORAL BIOAVAILABILITY OF LIPOPHILIC COMPOUND

(71) Applicant: Avantsar Sdn Bhd, Miri (MY)

(72) Inventors: Eric Kwong Seng Kiu, Miri (MY); Sheau Chin Lim, Kuching (MY)

(73) Assignee: Avantsar Sdn Bhd, Miri (MY)

(21) Appl. No.: 18/578,252

(22) PCT Filed: Oct. 31, 2022
(86) PCT No.: PCT/MY22/50101

§ 371 Date: Jan. 10, 2024

(30) Foreign Application Priority Data
Jul. 6, 2022 (MY) PI2022003597

(51) Int. Cl. A61K 31/353; A61K 9/00; A61K 9/107; A61K 47/22; A61K 47/24; A61K 47/44

(52) U.S. Cl. CPC A61K 31/353; A61K 9/0053; A61K 9/107; A61K 47/22; A61K 47/24; A61K 47/44

(57) ABSTRACT

The present invention relates to a self-emulsifying drug delivery formulation with improved oral bioavailability of lipophilic compound comprising a lipophilic compound, a Vitamin E polyethylene glycol 1000 succinate (TPGS), an oil carrier and a phospholipid. The present invention also relates to the use of the self-emulsifying drug delivery system in the manufacture of a dietary supplement with improved oral bioavailability of lipophilic compound.

### FIELD OF INVENTION

The present invention relates generally to a drug delivery system for lipophilic compounds, more particularly to a self-emulsifying drug delivery system with improved oral bioavailability of lipophilic compounds.

### BACKGROUND OF THE INVENTION

Vitamin E is the collective name for lipophilic, naturally occurring compounds with distinctive antioxidant properties normally found in vegetation and seeds. Naturally occurring vitamin E exists in eight chemical isomers, namely alpha-, beta-, gamma-, and delta-tocopherol and alpha-, beta-, gamma- and delta-tocotrienol, each having varying levels of biological activity. It is well known in the art that antioxidants protect cells from oxidative damage due to free radicals that contain an unpaired electron, and are widely attributed to the development of cancer and cardiovascular diseases. As such, vitamin E emerged as an essential lipophilic nutrient that possesses antioxidant properties for prevention of numerous diseases and promote health in the human body. Up until recently, the alpha isomer of tocopherol has been regarded as the most active form that is recognized to meet human requirements. However, years of scientific research found that tocotrienols are distinguished from the commonly used vitamin E isomer and suggests properties that are stronger than tocopherols. Tocotrienols are generally distributed throughout the human body via the bloodstream and tend to accumulate in body tissues such as the brain, heart, cardiac muscle, skin, liver and adipose tissue after oral administration. This indicates that the tocotrienol subfamily of vitamin E has better antioxidant properties than tocopherol subfamily as it show powerful neuroprotective, tumour suppressive and cholesterol lowering properties.

Like all lipophilic nutrients and dietary lipids, the oral absorption of tocotrienols is increased with the increased intake of fat due to the secretion of bile acids in order to facilitate lipolysis and formation of micelles for transport across the intestinal barrier. Unlike tocopherols which are ubiquitous in dietary sources, tocotrienols are limited in natural dietary sources and have lower affinity towards some transport proteins. When administered orally, tocotrienols tend to compete with tocopherols for alpha-tocopherol transport proteins (α-TTP) with approximately 10-fold lower affinity than with alpha-tocopherol. Hence, tocotrienols typically have low or erratic oral bioavailability.

To circumvent the issues of low or erratic oral bioavailability and to achieve consistently high absorption of lipophilic vitamins including tocotrienols, emulsions have been considered to improve thereof. However, conventional emulsions are not a desirable dosage form as they are relatively bulky, have shorter shelf life due to stability issues and are less palatable. Recently, there is a great attention in developing self-emulsifying drug delivery systems (SEDDS) that promise enhanced bioavailability, improved reproducibility of plasma profiles and reduced inter- and intra-subject variability. SEDDS are generally formulated in the absence of water by mixing oil with suitable non-ionic surfactants such that lipophilic drugs and compounds having sufficient solubility in the oil/surfactant system can be encapsulated therein.

Existing SEDDS technologies for effective delivery of tocotrienols have been proposed. For example, U.S. Pat. No. 6,596,306B1 discloses a self-emulsifying drug delivery composition for use with oral administration of fat-soluble drugs including tocotrienols. The aforementioned technology promises enhanced oral absorption of tocotrienols by employing a suitable combination of a surfactant system of caprylocaproyl macrogolglycerides and polyoxyethylene 20 sorbitan monooleate with an appropriate oil to promote self-emulsification and thereby further increasing the absorption of tocotrienols. Another SEDDS technology is exemplified in U.S. Pat. No. 10,493,055B2 that discloses a self-emulsifying drug delivery formulation for improved delivery of tocotrienols that comprises tocotrienols, a combination of two non-ionic surfactants, namely sorbitan monolaurate and polyoxyethylene sorbitan 20 monooleate, and an oil carrier. Nevertheless, the aforementioned SEDDS technologies seem to report at least 2 to 3-folds of bioavailability improvement that that of conventional non-self-emulsifying formulations.

Tocotrienols which are not rapidly be transported out of the liver will undergo catabolic metabolism by cytochrome P450 (CYP) enzyme, followed by P-oxidation, conjugation to generation carboxychromanols and conjugated counterparts. Tocotrienols, at very high concentration, could potentiate the transfer activity of P-glycoprotein in intestinal cells. However, such teaching is not apparent to persons skilled in the art to consider the impact of tocotrienols on the P-glycoprotein efflux mechanisms to promote bioavailability of tocotrienols with another inhibitor.

It has been reported that some surfactants also possess modulatory properties towards cytochrome P450 enzyme metabolism of drugs. Earlier uses of surfactants with tocotrienols such as those depicted in the abovementioned technologies gave no indication of possible concurrent inhibition of P-glycoprotein and CYP-mediated metabolism could complement and amplify the performance of a self-emulsifying formulation on the oral bioavailability of tocotrienols. In particular, alpha-tocopheryl polyethylene glycol 1000 succinate or TPGS is a non-ionic surfactant that is able to improve lipophilic drug solubility and enhance drug permeation due to its P-glycoprotein inhibition effect. Additionally, TPGS has been demonstrated to improve drug stability by inhibiting CYP3A4 and CYP2C9 metabolism. However, earlier uses of TPGS gave no indication that TPGS could amplify the oral bioavailability of another vitamin E isomer such as tocotrienol which competes with tocopherol for α-TTP, by way of a triple-action mechanism of spontaneous micellization, inhibition of P-gp and CYP-mediated metabolism. The present invention prompts the employment of TPGS in a self-emulsifying drug delivery formulation to trigger the aforementioned synergistic effect which could amplify the oral bioavailability of lipophilic tocotrienols.

### SUMMARY OF INVENTION

One aspect of the invention is to provide a self-emulsifying drug delivery formulation which exhibits enhanced oral bioavailability of lipophilic compounds upon oral ingestion. Advantageously, the self-emulsifying drug delivery formulation of the present invention exhibits enhanced oral bioavailability by 2 to 3 folds compared to conventional self-emulsifying drug delivery formulations.

Another aspect of the invention is to provide a self-emulsifying drug delivery formulation that is substantially stabilized.

At least one of the preceding objects is met, in whole or in part, in which the embodiment of the present invention describes a self-emulsifying formulation with improved oral bioavailability of lipophilic compound comprising a lipophilic compound, a Vitamin E polyethylene glycol 1000 succinate (TPGS), an oil carrier and a phospholipid.

In a preferred embodiment of the present invention, it is disclosed that the lipophilic compound is selected from the group consisting of tocotrienols, coenzyme Q10, fat-soluble vitamins, carotenoids or a combination of two or more thereof.

Preferably, the tocotrienols further comprise alpha-tocotrienol, beta-tocotrienol, gamma-tocotrienol or delta-tocotrienol.

Preferably, the coenzyme Q10 further comprises ubiquinone and ubiquinol.

Preferably, the fat-soluble vitamins are selected from Vitamin A, Vitamin D, Vitamin E, Vitamin K or a combination of two or more thereof.

Preferably, the carotenoids further comprise alpha-carotene, beta-carotene, beta-cryptoxanthin, lutein, zeaxanthin and lycopene.

In a preferred embodiment of the present invention, it is disclosed that the Vitamin E TPGS is present in an amount ranging from 0.1% to 30% by weight of the drug delivery formulation.

In another preferred embodiment of the present invention, it is disclosed that the oil carrier is selected from the group consisting of fatty acid esters of glycerol, fatty acid esters of propylene glycol, vegetable oils or a combination or two or more thereof.

Preferably, the fatty acid ester of glycerol is monoglyceride, diglyceride or triglyceride.

Preferably, the vegetable oil is palm olein, soybean oil, sesame oil, rice bran oil, sunflower oil or castor oil.

More preferably, the oil carrier is present in an amount ranging from 5% to 80% by weight if the drug delivery formulation.

Further embodiment of the present invention discloses that the phospholipid is lecithin comprising phosphatidylcholine, phosphatidylethanolamine and phosphatidylinositol.

Preferably, the phospholipid is present in an amount ranging from 1% to 10% by weight of the drug delivery formulation.

It is preferred that the drug delivery formulation is in the form of a capsule or softgel.

An exemplary embodiment of the present invention discloses the use a self-emulsifying drug delivery formulation in the manufacture of a dietary supplement with improved oral bioavailability of lipophilic compound, wherein the formulation comprises a lipophilic compound selected from the group consisting of tocotrienols, coenzyme Q10, fat-soluble vitamins, carotenoids or a combination of two or more thereof, the lipophilic compound present in an amount ranging from 5% to 80% by weight of the drug delivery formulation, a Vitamin E polyethylene glycol 1000 succinate (TPGS) present in an amount ranging from 0.1% to 30% by weight of the drug delivery formulation, an oil carrier and a phospholipid.

One skilled in the art will readily appreciate that the present invention is well adapted to carry out the objects and obtain the ends and advantages mentioned, as well as those inherent therein. The embodiment described herein is not intended as limitations on the scope of the invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, its construction and operation and many of its advantages would be readily understood and appreciated.

FIG. 1 depicts the oral bioavailability of delta-tocotrienol with and without single dose of ketoconazole represented in a blood concentration-time curve.

FIG. 2 depicts the oral bioavailability of gamma-tocotrienol with and without single dose of ketoconazole represented in a blood concentration-time curve.

FIG. 3 depicts the oral bioavailability of alpha-tocotrienol with and without single dose of ketoconazole represented in a blood concentration-time curve.

FIG. 4 depicts the oral bioavailability of delta-tocotrienol administered via the Control, Product X and Formulation S represented in a blood concentration-time curve.

FIG. 5 depicts the oral bioavailability of gamma-tocotrienol administered via the Control, Product X and Formulation S represented in a blood concentration-time curve.

FIG. 6 depicts the oral bioavailability of alpha-tocotrienol administered via the Control, Product X and Formulation S represented in a blood concentration-time curve.

### DETAILED DESCRIPTION OF THE INVENTION

Hereinafter, the invention shall be described according to the preferred embodiments of the present invention and by referring to the accompanying description and drawings. However, it is to be understood that limiting the description to the preferred embodiments of the invention is merely to facilitate discussion of the present invention and it is envisioned that those skilled in the art may devise various modifications without departing from the scope of the appended claim.

The present invention provides a formulation for a self-emulsifying drug delivery system (SEDDS) for lipophilic compounds with an enhanced oral bioavailability and consistent high amount of absorption of the lipophilic compounds. SEDDS are essentially mixtures of oil and surfactants and have been widely used for lipophilic-based formulations. SEDDS forms oil-in-water emulsions upon exposure to gastrointestinal fluids with gentle agitation such as peristaltic movement of stomach and small intestine. As such, SEDDS efficiently provides improvement in absorption and oral bioavailability. The selection of the suitable combination of oil carriers and surfactants to achieve consistently high oral bioavailability of lipophilic compounds is an essential requirement for formulation of SEDDS as the solubility and efficiency of oral bioavailability of the lipophilic compounds from the SEDDS are determined by the selection of oil carriers and surfactants.

In an embodiment of the present invention, the SEDDS formulation comprises a lipophilic compound, a surfactant, an oil carrier and a phospholipid. By definition, a lipophilic compound is a molecule that is attracted to and tends to dissolve in other lipids, fats and oils. Any suitable lipophilic compound may be chosen by one of skill in the art. Lipophilic compounds appropriate for use in the SEDDS formulation of the present invention is selected from the group consisting of tocotrienols, coenzyme Q10, fat-soluble vitamins, carotenoids or a combination of two or more thereof. The lipophilic compounds used in the SEDDS formulation of the present invention can provide additional desirable nutritional or pharmaceutical benefits in combination with the benefits provided by the surfactant, oil carrier and phospholipid within the formulation, and can be chosen accordingly as desired. It is preferred that the lipophilic compound is present in an amount ranging from 5% to 80% by weight of the SEDDS formulation.

According to a preferred embodiment of the present invention, the tocotrienols used in the SEDDS formulation further comprise alpha-tocotrienol, beta-tocotrienol, gamma-tocotrienol or delta-tocotrienol. The tocotrienols used in the SEDDS formulation of the present invention can be derived naturally or synthetically. For example, the tocotrienols may be extracted from plants such as palm oil, bran oil, flaxseed oil, wheat germ, barley and certain types of nuts and grains, but not limited thereto.

In another embodiment of the present invention, the lipophilic compound used in the SEDDS formulation is coenzyme Q10. As used herein, coenzyme Q10 is a fat-soluble quinone with a structure similar to that of Vitamin K and possesses antioxidant properties. Suitable coenzyme Q10 used in the SEDDS formulation of the present invention includes ubiquinone and ubiquinol.

In another embodiment of the present invention, the lipophilic compound used in the SEDDS formulation is fat-soluble vitamins. Fat-soluble vitamins are essentially vitamins that are dissolved in fats and are absorbed by fat globules that travel through the small intestines and distributed through the body in the bloodstream. In the embodiment of the present invention, the fat-soluble vitamin used in the SEDDS formulation includes, but not limited to, Vitamin A, Vitamin D, Vitamin E and Vitamin K.

In another embodiment of the present invention, the lipophilic compound used in the SEDDS formulation is carotenoids. Generally, carotenoids are plant pigments responsible for bright red, yellow and orange hues in fruits and vegetables. Carotenoids are a class of phytonutrients found in the cells of a wide variety of plants, algae and bacteria. Carotenoids also act as antioxidants in the human body. Suitable carotenoids used in the SEDDS formulation of the present invention include, but not limited to, alpha-carotene, beta-carotene, beta-cryptoxanthin, lutein, zeaxanthin and lycopene.

The SEDDS formulation of the present invention comprises the abovementioned lipophilic compounds in combination with a surfactant and an oil carrier as useful excipients to provide a self-emulsifying feature. The solubility of the lipophilic compound in a lipid system can be greatly increased by mixing the lipophilic compound with an acceptable oil carrier, and the mixture thereof can be readily emulsified with a surfactant. Conventionally, surfactant commonly used in a SEDDS formulation includes a non-ionic surfactant that has a hydrophobic component (oil soluble) and a hydrophilic (water soluble) and is characterized by their hydrophilic-lipophilic balance (HLB). Non-ionic surfactant is provided to reduce the interfacial tension between oil and water by adsorbing at the interface between oil and water A variety of pharmaceutically acceptable surfactants are suitable for use in the production of a SEDDS formulation.

Conventional SEDDS formulations reported improvements of oral bioavailability by 2 to 5 folds compared to non-SEDDS formulations. The inventors found out that potential limitation of the intestinal first-pass effects via inhibition on both efflux transport protein of P-glycoprotein and metabolizing enzyme of CYP3A4 enzyme improved the oral bioavailability of the lipophilic compounds by an additional 2 to 3 folds compared to the improvement conferred by conventional SEDDS formulation. In the context of the present invention, the synergistic effect of inhibition of P-glycoprotein and CYP3A4 enzyme can be potentiated by employing a water soluble derivative of natural Vitamin E, namely Vitamin E polyethylene glycol 1000 succinate (TPGS). TPGS can improve solubility of the lipophilic compounds in the SEDDS formulation of the present invention and enhance permeation thereof due to its P-glycoprotein inhibition effect. Further, TPGS have been demonstrated to improve compound stability by inhibiting CYP3A4 metabolism. TPGS is also an excellent emulsifier for lipophilic compounds and an enhancer of oral bioavailability in a SEDDS formulation by forming stable emulsions of small particle size. As used herein, the term “TPGS” is used interchangeably with the term Vitamin E TPGS. According to a preferred embodiment of the present invention, the Vitamin E TPGS used is present in an amount ranging from 0.1% to 30% by weight of the SEDDS formulation, depending on the lipophilic compound used therein.

As abovementioned, the SEDDS formulation comprises an oil carrier in combination with Vitamin E TPGS to provide a self-emulsifying feature. Upon agitation, the oil carrier exhibits small oil droplets which are then dispersed evenly and self-assembled into micelles in the presence of the Vitamin E TPGS. In a preferred embodiment of the present invention, the oil carrier used in the SEDDS formulation is selected from the group consisting of fatty acid esters of glycerol, fatty acid esters of propylene glycol, vegetable oils or a combination of two or more thereof. Preferably, the fatty acid ester of glycerol used is monoglyceride, diglyceride or triglyceride. Suitable vegetable oil used as the carrier oil in the SEDDS formulation of the present invention includes, but not limited to, palm olein, soybean oil, sesame oil, rice bran oil, corn oil, sunflower oil or castor oil. To provide a sufficient self-emulsifying feature of the SEDDS formulation, the oil carrier used is present in an amount ranging from 5% to 80% by weight of the SEDDS formulation. It is to be noted that the amount of the oil carrier may be adjusted depending on the amount of Vitamin E TPGS used in the SEDDS formulation.

Another excipient used in the SEDDS formulation of the present invention is phospholipids, more preferably plant-based phospholipids. It was found that the combination of phospholipids with the oil carrier significantly exerts stabilizing effects in the micellar structure of the SEDDS formulation for delivering the lipophilic compound. In this way, the oral bioavailability of the lipophilic compound can be enhanced. In a preferred embodiment of the present invention, the phospholipid used is lecithin comprising phosphatidylcholine, phosphatidylethanolamine and phosphatidylinositol. Preferably, the phospholipid used is present in an amount ranging from 1% to 10% by weight of the SEDDS formulation.

In an embodiment of the present invention, the SEDDS formulation is prepared by adding one or more lipophilic compounds to Vitamin E TPGS, which are heated to about 45° C. to 50° C. A suitable oil carrier and phospholipid are then added into the mixture and continuously mixed for about 30 minutes to obtain a homogenous mixture. To obtain a SEDDS formulation of the present invention, the amounts of lipophilic compounds, Vitamin E TPGS, oil carrier and phospholipid are provided in the abovementioned. Thereinafter, the homogenous mixture is cooled to room temperature and then filled into any suitable oral dosage form. In a preferred embodiment of the present invention, the SEDDS formulation is encapsulated into hard capsules or softgel capsules.

In accordance to that, an exemplary embodiment of the present invention therefore discloses the use of the SEDDS formulation in the manufacture of a dietary supplement with improved oral bioavailability of lipophilic compound. It is to be understood that the SEDDS formulation of the present invention may be combined with, and may comprise, flavourings, colorants, excipients, stabilizers and other agents known to any person skilled in the art of dietary supplement formulation.

### EXAMPLE

The following non-limiting examples have been carried out to illustrate the preferred embodiments of the invention.

### Example 1

A 2-period, 2-sequence crossover study was conducted using rats to investigate the impact of co-administration of ketoconazole, a strong inhibitor of P-glycoprotein and CYP3A4 enzyme on the oral bioavailability of tocotrienols (T3), a substrate of P-glycoprotein and CYP3A4 enzyme. The animals were randomly divided into 2 groups, and administered preparations following the sequence shown in Table 1 below:

TABLE 1

Sequence of administration

| Group | Phase I | Phase II |
|---|---|---|
| 1 | T3 Without ketoconazole | T3 With ketoconazole |
| | (control) | |
| 2 | T3 With ketoconazole | T3 Without ketoconazole |
| | | (control) |

Non self-emulsifying tocotrienol oily suspension was prepared by mixing tocotrienols rich fraction (TRF) and palm olein at a ratio of 1:1. The total tocotrienol administered per rat was equivalent to 10 mg/kg body weight. The single dose of ketoconazole (suspension in water) was administered at a dose of 32 mg/kg body weight.

The rats were fasted overnight for at least 12 hours before the study. During Phase 1, the rats in Group 1 were given only the tocotrienol oily suspension while Group 2 were given a single dose of ketoconazole half an hour prior to administration of tocotrienol. Phase 2 was carried out after 1 week of wash-out period. Blood samples were taken from the tail vein according to the following predetermined sampling interval. The blood levels of individual tocotrienol isomers at each time point was analysed using HPLC assay.

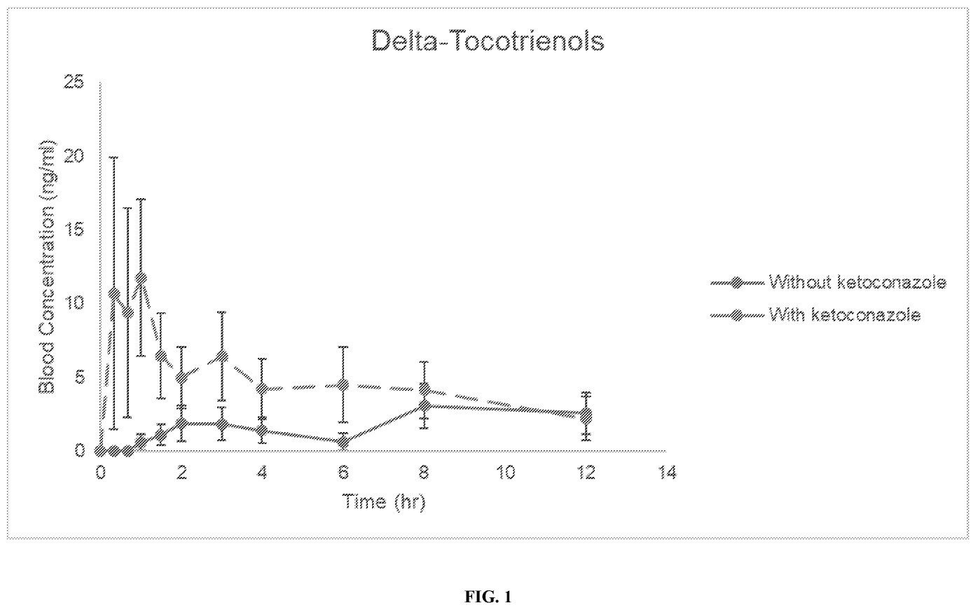

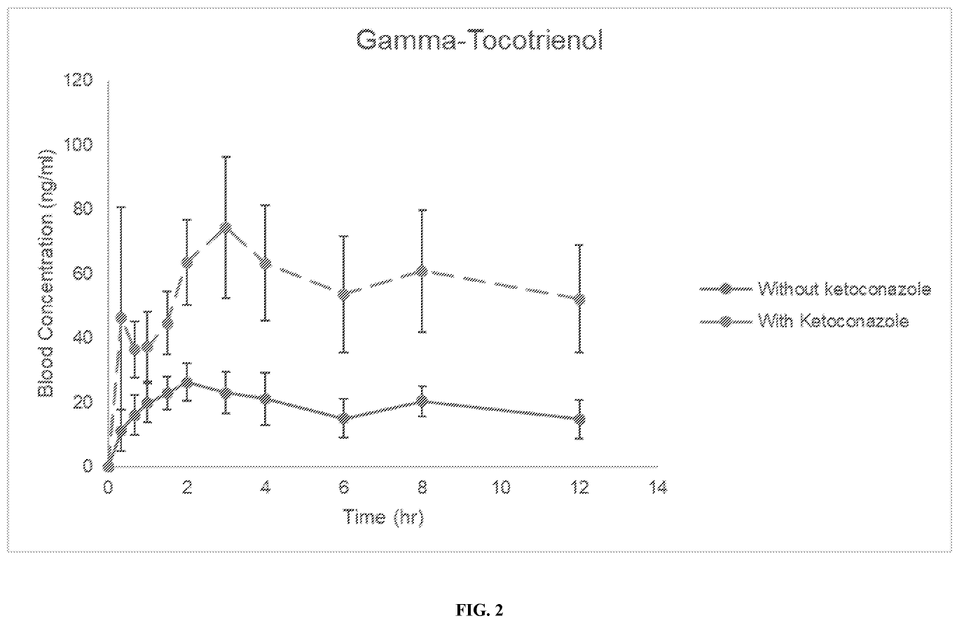

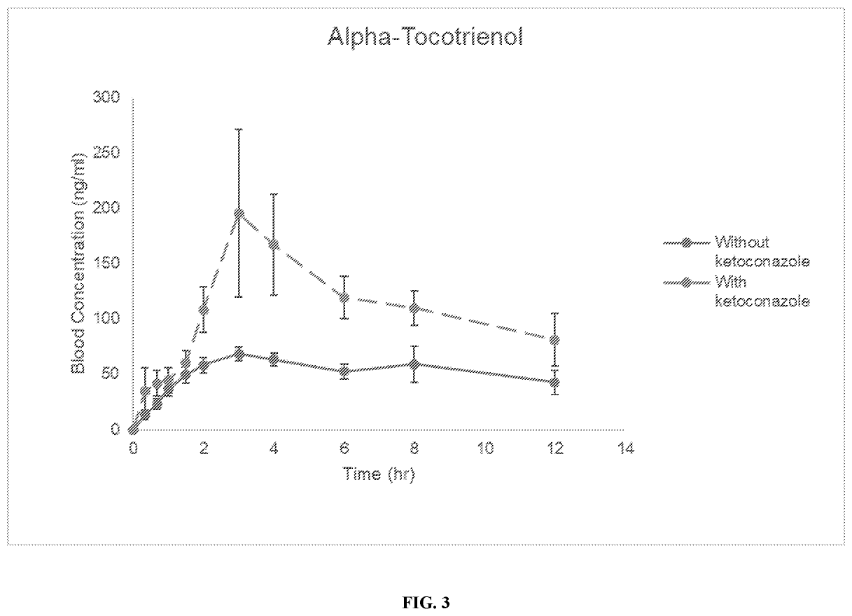

The impact of CYP3A4 enzyme and P-glycoprotein inhibition was confirmed on the oral bioavailability of tocotrienol. The co-administration of ketoconazole (strong inhibitor of P-glycoprotein and CYP3A4 enzyme) increased the oral bioavailability of tocotrienols in rats by approximately 2.4-fold. Table 2 summarizes the improvements in oral bioavailability of the individual tocotrienol isomers compared with control. FIGS. 1-3 depict the oral bioavailability of the individual tocotrienol isomers with and without single dose of ketoconazole represented in a blood concentration-time curve.

TABLE 2

| | Increase in bioavailability compared with Control | | | |
|---|---|---|---|---|
| | Delta- | Gamma- | Alpha- | Total |
| | tocotrienol | tocotrienol | tocotrienol | tocotrienols |
| With single | 2.6-fold | 3.0-fold | 2.1-fold | 2.4-fold |
| dose of | | | | |
| Ketoconazole | | | | |

Typically, a compound can be determined as a substrate of P-glycoprotein and CYP3A4 enzyme if it achieves a significantly enhanced oral bioavailability with co-administration of a strong inhibitor such as ketoconazole—to inhibit the efflux transport by P-glycoprotein and metabolism by CYP enzymes. In this experiment, the administration of single dose of ketoconazole prior to dosing tocotrienol preparation resulted in approximately 2.4-fold increase in total tocotrienols detected in the blood. Thus, it can be concluded that tocotrienols are substrates of P-glycoprotein and CYP3A4 enzyme.

### Example 2

A 3-period, 3-sequence crossover study was conducted using rats to compare the oral bioavailability of tocotrienols in Control (non self-emulsifying oily suspension), Product X (commercial self-emulsifying formulation) and Formulation S (the self-emulsifying drug delivery formulation of the present invention). The animals were randomly divided into 3 groups and were administered the preparations following the sequence shown in Table 3 below.

TABLE 3

Sequence of administration

| Group | Phase I | Phase II | Phase III |
|---|---|---|---|
| 1 | Control | Formulation S | Product X |
| 2 | Formulation S | Product X | Control |
| 3 | Product X | Control | Formulation S |

The Control (non self-emulsifying oily suspension) was prepared by mixing tocotrienols rich fraction (TRF) and palm olein at a ratio of 1:1.

Product X (self-emulsifying commercial product) contains tocotrienols formulated with a patented self-emulsifying system for enhanced oral bioavailability.

Formulation S comprises tocotrienols:oil carrier at about 5-80% by weight, and the TPGS:phospholipid phase at about 0.1 to 30% by weight. The ratio of tocotrienols to oil carrier may range from 1:1 to 1:10. The ratio of the TPGS and phospholipid may range from 1:1 to 1:20.

The rats were fasted overnight for at least 12 hours before the study. During Phase 1, rats in Group 1 were given T3 oily suspension (non self-emulsifying control), Group 2 were given Formulation S, and Group 3 was given commercial Product X. The total tocotrienols administered per rat for each preparation was equivalent to 10 mg/kg body weight. Phase 2 and 3 were carried out after one week of wash-out period. Blood samples were taken from the tail vein according to the following a predetermined sampling interval. The blood levels of individual tocotrienol isomers at each time point was analyzed using HPLC assay.

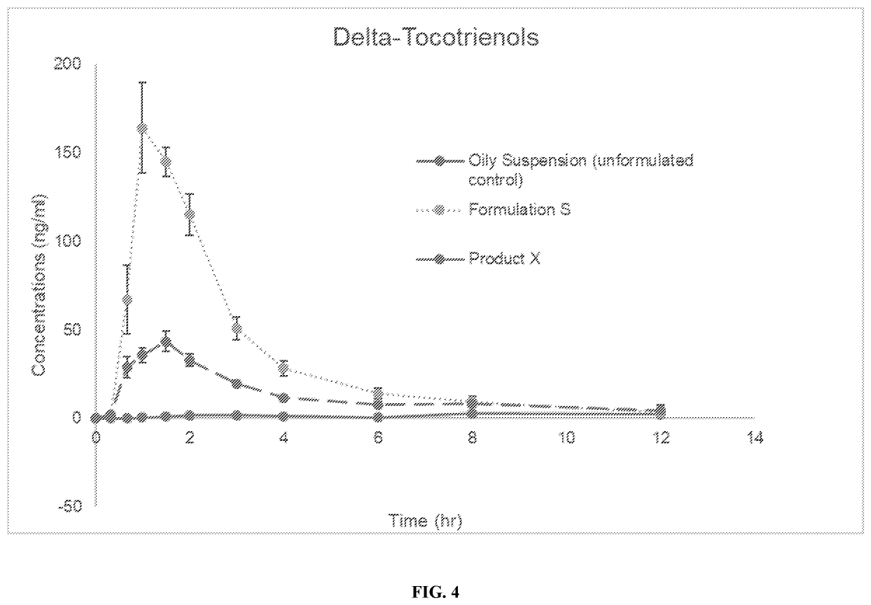

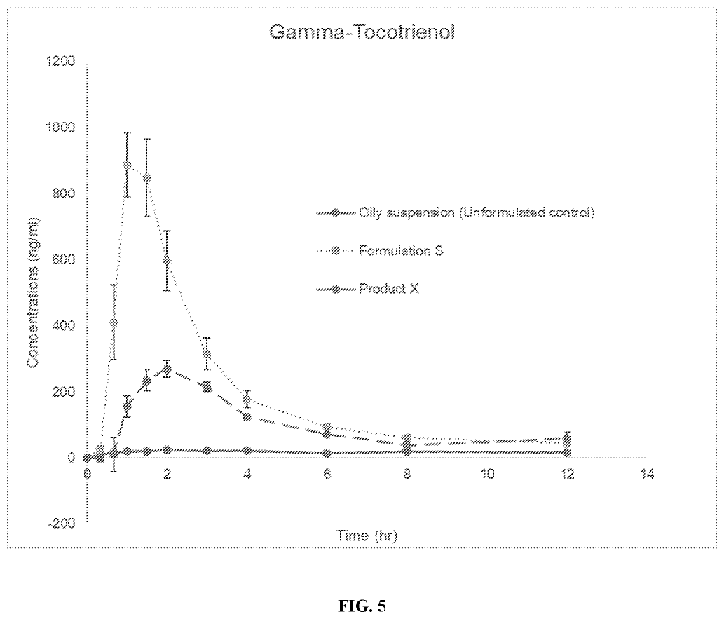

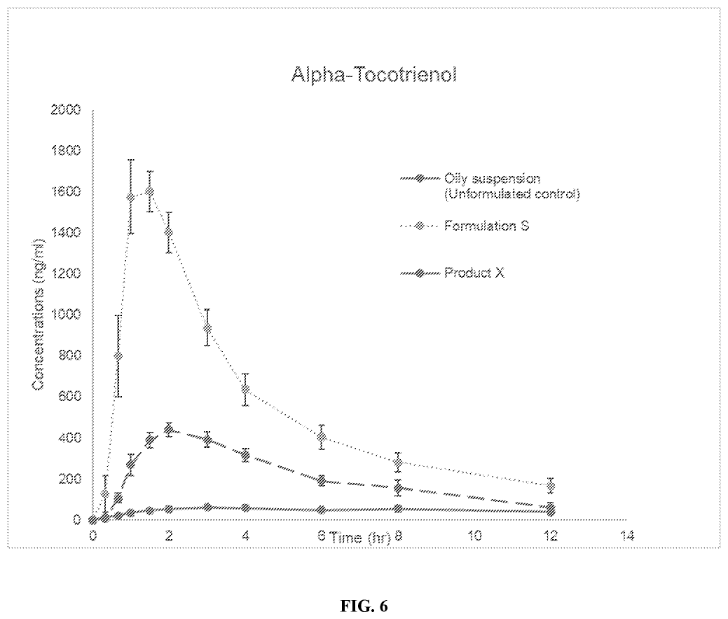

Compared to the non self-emulsifying control, the commercial Product X increased the oral bioavailability of total tocotrienols by approximately 4.6-folds, while Formulation S increased the oral bioavailability by 11.5-folds. Formulation S demonstrated a synergistic effect from the combination of the excipients to yield a super-enhanced oral bioavailability which far exceeded the bioavailability improvement observed with simple inhibition of the metabolizing enzymes using ketoconazole, and also outperformed the bioavailability improvement by simple self-emulsifying formulation seen in Product X. Table 4 summarizes the improvements in oral bioavailability of the individual and total tocotrienol isomers compared with control, Product X and Formulation S. FIGS. 4-6 depict the oral bioavailability of the individual tocotrienol isomers administered via the Control, Product X and Formulation S represented in a blood concentration-time curve.

TABLE 4

| | Increased bioavailability compared to Control | | | |
|---|---|---|---|---|
| | Delta- | Gamma- | Alpha- | Total |
| | tocotrienol | tocotrienol | tocotrienol | tocotrienols |
| With a single | 2.6-fold | 3.0-fold | 2.1-fold | 2.4-fold |
| dose of | | | | |
| Ketaconazole | | | | |
| With Product | 8.5-fold | 4.8-fold | 4.4-fold | 4.6-fold |
| X | | | | |
| With | 21.1-fold | 10.7-fold | 11.4-fold | 11.5-fold |
| Formulation | | | | |
| S | | | | |

Self-emulsifying formulation is a common strategy to improve oral bioavailability of oil-soluble compounds. The typical enhancement of self-emulsifying preparations as described in literature is 2 to 5 folds compared to non self-emulsifying preparation. Example 1 above suggested that tocotrienols, being substrate of P-glycoprotein and CYP3A4 enzyme could achieve enhanced oral bioavailability with the inhibition of P-glycoprotein and CYP enzyme using ketoconazole. The recorded enhancement of bioavailability is 2 to 3 folds greater compared to without ketoconazole.

In the present invention, Formulation S contained TPGS:phospholipid combination with oil-soluble tocotrienols which promoted spontaneous micellization, limited the intestinal first-pass effects via inhibition of P-glycoprotein and CYP3A enzyme metabolism, and stabilized the emulsion to result in greater intestinal absorption of tocotrienols upon oral administration. The bioavailability enhancement of the present invention far exceeded expected performance (by additive effect) of individual strategies, namely co-administration of inhibitor/ketoconazole alone or by conventional self-emulsifying preparation alone. With this invention, the oral bioavailability of tocotrienols was enhanced by 11.5-folds compared to non self-emulsifying oily preparation of tocotrienols, which has not been recorded before.

### Example 3

The test apparatus used for evaluating the self-emulsifying efficiency of the present invention consisted of a light source, a paddle stirrer, a 250 ml beaker, a current relay and a phototransistor, which were aligned accordingly. The light source was from a 40-watt bulb, giving light intensity of about 1000 lux that pass through the glass beaker filled with 250 ml distilled water. The phototransistor was connected to a current relay and a stopwatch. The paddle stirrer was set to rotate at 100 rpm.

To assess the self-emulsifying properties of the present invention, a 1 ml syringe containing 0.5 ml of the liquid formulation was placed 1 cm below the water surface in the beaker prior to injection. When the sample was introduced into the 250 ml distilled water (37° C.), the stopwatch was initiated simultaneously. The paddle stirrer provided gentle agitation to the contents in the beaker. If an emulsion formed and blocked the transmission of light through the beaker, the phototransistor would not be able to detect any light and the stopwatch would be triggered to stop via the current relay. The time recorded was used to compare the self-emulsifying efficiency among the different samples as shown in Table 5 below:

TABLE 5

| | A | | | |
|---|---|---|---|---|
| Formulation | (Product X) | B | C | D |
| Time | 3.8 ± 0.2 | 3.9 ± 0.3 | 3.8 ± 0.4 | 3.9 ± 0.2 |
| (seconds) | | | | |

Note:
n time in seconds (mean ± SD, n = 3)

The physical stability of the emulsion products formed after standing for 20 minutes at room temperature (25° C.) were evaluated. An amount (1 ml) of each formulation was dispersed into 50 ml of distilled water and rotated for 5 minutes on a rotator. The test tubes were left standing and visually inspected. Physical stability of the emulsion products based on visual observation was categorized as no separation, slight creaming, creaming and complete separation of phases, as summarized in Table 6 below. All experiments were conducted in triplicates.

TABLE 6

| | Time | | | |
|---|---|---|---|---|
| | 5 | 10 | 15 | 20 |
| Formulation | min | min | min | min |
| A | No | Slight | Slight | Creaming |
| (Product X) | separation | creaming | creaming | |
| B | No | No | No | No |
| | separation | separation | separation | separation |
| C | No | No | No | No |
| | separation | separation | separation | separation |
| D | No | No | No | No |
| | separation | separation | separation | separation |

The self-emulsifying efficiency remained comparable for Formulation A (Product X) versus Formulation B, C, and D (Formulation B being the final formulation tested in the animal study of Example 2). The comparable results show that when the formulation was dispersed in water, the formulation self-emulsified almost immediately, as determined by the time required to block the light transmission in the first part of this experiment.

However, when the self-emulsified formulations left to stand over time, there are significant differences in the stability of the self-emulsified state of the formulation. Formulation A (Product X) started to show slight creaming at 10 minutes (indicating the beginning of separation), while Formulation B, C and D remained stable with no separation even up to 20 minutes.

The present disclosure includes as contained in the appended claims, as well as that of the foregoing description. Although this invention has been described in its preferred form with a degree of particularly, it is understood that the present disclosure of the preferred form has been made only by way of example and that numerous changes in the details of construction and the combination and arrangements of parts may be resorted to without departing from the scope of the invention.

1.-16. (canceled)

17. A self-emulsifying drug delivery formulation with improved oral bioavailability of lipophilic compound, comprising:
a lipophilic compound comprising a tocotrienol, coenzyme Q10, a carotenoid or a combination of two or more thereof;
a Vitamin E polyethylene glycol 1000 succinate (TPGS);
an oil carrier; and
a phospholipid.

18. The drug delivery formulation according to claim 17, wherein the tocotrienol is selected from alpha-tocotrienol, gamma-tocotrienol, delta-tocotrienol or a combination of two or more thereof.

19. The drug delivery formulation according to claim 17, wherein the coenzyme Q10 is selected from ubiquinone, ubiquinol or a combination thereof.

20. The drug delivery formulation according to claim 17, wherein the carotenoid is selected from alpha-carotene, beta-carotene, beta-cryptoxanthin, lutein, zeaxanthin, lycopene or a combination of two or more thereof.

21. The drug delivery formulation according to claim 17, wherein the lipophilic compound further comprises fat-soluble vitamins.

22. The drug delivery formulation according to claim 21, wherein the fat-soluble vitamins are selected from Vitamin A, Vitamin D, Vitamin E, Vitamin K or a combination of two or more thereof.

23. The drug delivery formulation according to claim 17, wherein the lipophilic compound is present in an amount ranging from 5% to 80% by weight of the drug delivery formulation.

24. The drug delivery formulation according to claim 17, wherein the Vitamin E TPGS is present in an amount ranging from 0.1% to 30% by weight of the drug delivery formulation.

25. The drug delivery formulation according to claim 17, wherein the oil carrier is selected from the group consisting of fatty acid esters of glycerol, fatty acid esters of propylene glycol, vegetable oils or a combination of two or more thereof.

26. The drug delivery formulation according to claim 25, wherein the fatty acid ester of glycerol is a monoglyceride, diglyceride or triglyceride.

27. The drug delivery formulation according to claim 25, wherein the vegetable oil is palm olein, soybean oil, sesame oil, rice bran oil, corn oil, sunflower oil or castor oil.

28. The drug delivery formulation according to claim 17, wherein the oil carrier is present in an amount ranging from 5% to 80% by weight of the drug delivery formulation.

29. The drug delivery formulation according to claim 17, wherein the phospholipid is lecithin comprising phosphatidylcholine, phosphatidylethanolamine and phosphatidylinositol.

30. The drug delivery formulation according to claim 17, wherein the phospholipid is present in an amount ranging from 1% to 10% by weight of the drug delivery formulation.

31. The drug delivery formulation according to claim 17, wherein the drug delivery formulation is in a form of a capsule or softgel.

32. Use of a self-emulsifying drug delivery formulation in the manufacture of a dietary supplement with improved oral bioavailability of lipophilic compound, wherein the formulation comprises a lipophilic compound selected from the group consisting of tocotrienols, coenzyme Q10, fat-soluble vitamins, carotenoids or a combination of two or more thereof, the lipophilic compound present in an amount ranging from 5% to 80% by weight of the drug delivery formulation, a Vitamin E polyethylene glycol 1000 succinate (TPGS) present in an amount ranging from 0.1% to 30% by weight of the drug delivery formulation, an oil carrier and a phospholipid.
